(19) United States
(12) Patent Application Publication
(10) Pub. No.: US 2009/0234429 A1
(43) Pub. Date: Sep. 17, 2009

(54) SELF-RESTRAINING ENDOLUMINAL PROSTHESIS

(76) Inventor: Lilip Lau, Sunnyvale, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/368,077

(22) Filed: Feb. 9, 2009

Related U.S. Application Data
(60) Provisional application No. 61/026,972, filed on Feb. 7, 2008.

(51) Int. Cl. A61F 2/06

(52) U.S. Cl. 623/1.12; 623/1.15

(57) ABSTRACT

A self-expanding, self-restraining endoluminal prosthesis is configured to be compressible into a collapsed state, and restrained in the collapsed state by structures integrated with the stent, and without the use of external structure. Such restraining structures engage one another to hold the prosthesis in the collapsed state. Expansile elements of the prosthesis urge the prosthesis to automatically expand once released from the collapsed state. Thus, once the restraining structures, which hold the prosthesis in the collapsed state, are disengaged, the prosthesis automatically expands to a deployed state.

### CROSS-REFERENCE TO RELATED APPLICATIONS

This application claims the benefit of U.S. Provisional Application No. 61/026,972, which was filed on Feb. 7, 2008. The entirety of the priority application is hereby incorporated by reference. In particular, the priority application includes photographs and associated description of prototype prostheses. These photographs, particularly the photographs presented as FIGS. 5, 6, 8, 11, 12, 14, 29, 31 and 35, are expressly incorporated by reference into the present application.

### BACKGROUND OF THE INVENTION

1. Field of the Invention

The present invention relates to endoluminal prostheses.

2. Description of the Related Art

Endoluminal prostheses, such as stents, are often used to treat maladies in the vasculature of patients. For Example, for patients experiencing acute coronary syndromes, stents are often placed in coronary arteries to support the artery so as to restore and ensure blood flow through an otherwise fully or partially occluded segment of the artery. Multiple stent products are currently in use, including drug eluting stents (DES) and bare metal stents (BMS). Drug eluting stents slowly apply pharmacological therapy to, for example, inhibit neointimal growth, and are thus associated with lower restenosis rates.

Most stents currently used in the coronary arteries are balloon expandable (BE), including for example the Cypher™ stent from Johnson & Johnson. This means that the stent, typically a stainless steel stent, is mounted in a compacted configuration on the balloon portion of balloon catheter. The catheter is manipulated through the patient's vasculature until the balloon/stent is at the desired treatment location. The clinician then inflates the balloon, thus plastically deforming, enlarging and deploying the stent. Once the stent is deployed, the balloon is deflated and removed from the patient's vasculature.

Balloon expandable stents inherently recoil elastically upon deflation of the balloon. Thus, upon deployment of a BE stent, the balloon must be inflated beyond the desired final diameter of the stent to compensate for the recoil. Also, the cross-sectional shape of balloon expandable stents is limited by the balloon shape, which typically is round. Thus BE stents may not conform to oval or eccentric-cross-section arteries.

Another type of stent is referred to as a self-expanding (SE) stent, an example of which is the Radius™ stent marketed by SciMed Life Systems. The Radius SE stent is constructed of a shape memory alloy material such as Nitinol. Thus, even if the stent is substantially deformed and compacted, it will seek to expand to its fully-expanded state when freed from restraint. In practice, the Radius stent is compacted about a catheter and held within a sheath that prevents the stent from expanding. The catheter, with the stent/sheath in place, is advanced through the patient's vasculature to the desired treatment location and the sheath is then removed, thus freeing the stent to expand to a deployed condition. The self-expanding stent thus expands and engages the vessel without requiring a deployment balloon.

Although use of stents has generally been found to provide significant advantages to patients, there are areas for improvement. For example, although drug eluting stents generally have lower restenosis rates than bare metal stents, some studies indicate that drug eluting stents may have higher late stent thrombosis rates as compared to bare metal stents. A stent thrombosis refers to thrombus created because of the presence of the stent. Late stent thrombosis refers to appearance of such thrombus after a substantial time has passed, such as more than one or even two years, since the stent was placed.

Studies indicate a connection between stent thrombosis and malapposition of the stent. Malapposition refers to a lack of contact of one or more stent struts with the target vessel wall. Clinically, it is defined as when intravascular ultrasound (IVUS) detects blood flow between at least one strut of a stent and the vessel wall. Malapposition creates abnormal hemodynamics that can lead to thrombogenesis.

With most balloon expandable stents, when the stent is expanded into place the clinician will use a balloon size chosen to expand the stent beyond the target vessel size because the stent will inherently recoil after expansion. As such, the balloon purposely overstretches the vessel, possibly causing further damage to the vessel.

With the Radius self-expanding stent, since the stent expands as the sheath is removed, frictional interference between the sheath and the stent can adversely affect deployment, disrupting uniformity and positional accuracy of placement of the stent on the vessel wall. Further, Radius SE stent placement typically is done in conjunction with a balloon angioplasty or other treatment applied to the vessel wall before or after placement of the self-expanding stent, thus damaging the vessel wall and hindering vessel healing.

### SUMMARY OF THE INVENTION

Accordingly, there is a need in the art for an endoluminal prosthesis such as a stent that can be deployed without overstretching and damaging the vessel wall, but which can be simply and precisely placed. Further, there is a need in the art for a stent that avoids malapposition, and in particular avoids malapposition over time as conditions within the associated vessel may change.

Even if a stent is initially placed with proper or substantially proper apposition, late stent thrombosis is thought to be associated with the stent becoming malapposed over time. Loss of proper apposition over time can result from multiple causes, which have been considered by Applicant. For example, if the stent is placed while the vessel is undergoing a transient vasoconstriction, as is often the case during ST-segment elevation myocardial infarction (STEMI), the stent may be sized to fit the vessel's constricted state. Upon resolution of the STEMI event, vasoconstriction may cease, and the stent is then undersized, and thus malapposed. Also, during stent placement thrombus may interfere with placement of the stent. This situation is often the case during a STEMI event. Further, over time, thrombus may resolve, yet the stent will maintain its shape from when it was initially placed. With the thrombus removed from the blood vessel, the stent no longer fits appropriately, and is malapposed. Also, in some instances a stented vessel may positively (radially outwardly) remodel. When a vessel becomes larger with positive remodeling, a typical balloon-expandable stent does not correspondingly increase in size, thus resulting in stent malapposition.

The present specification describes stent embodiments that can be deployed into vessels experiencing transient conditions such as vasoconstriction, thrombus-formation and the like in order to open such vessels to blood flow. However, over time as conditions change within the vessel, the stent correspondingly changes, thus preventing malapposition over time.

Embodiments described herein are depicted in connection with a self-restraining, self-expanding stent for use in coronary arteries. In at least some embodiments a stent includes a plurality of pairs of latching members that, when engaged, hold the stent collapsed about a delivery device such as a PTCA catheter. The catheter is advanced through the patient's vasculature until the stent/balloon is positioned at a desired treatment point. The balloon is then inflated, thus defeating the engaged latching members and enabling the stent to expand from its collapsed state toward its natural, deployed state.

In some embodiments the deployment balloon is sized so that it expands sufficient to defeat the engaged latching member pairs so that the stent is no longer restrained. In some embodiments, the balloon is sized sufficient to also engage plaques or the like within the vessel, or even to engage the vessel wall so as to provided treatment and/or relief from full or partial vessel occlusion. In other embodiments, the balloon is sized and configured so that it does not actually engage the vessel wall, but leaves treatment entirely to the self-expanding stent.

In some embodiments a stent is provided that is sized to have a maximum diameter greater than the inner diameter of the target vessel so that it applies pressure and expands to fit the size of the vessel. Most preferably the stent has a maximum diameter slightly larger than the vessel. Thus, the stent will not be undersized even as conditions in the vessel change over time. For example, if thrombus that is in place when the stent is deployed later resolves, the stent will automatically expand into the area previously taken up by the thrombus, thus avoiding malapposition over time. Also, such a stent may automatically adjust to irregularities in the shape of the vessel as conditions within the vessel change over time.

The present specification describes several inventive aspects in connection with several embodiments. It is to be understood that the embodiments depicted herein are intended to be examples presenting inventive aspects in the context of a stent. Such inventive aspects can be applied in different configurations and in products other than stents.

In accordance with one embodiment, the present invention provides a self-restraining, self-expanding stent, comprising an elongate, unitarily-formed tubular body having a longitudinal axis and comprising a plurality of elastic expansile elements. The tubular body is configured to be collapsible radially inwardly into a compacted configuration, the elastic expansile elements urging the tubular body to expand radially outwardly toward an expanded configuration. First and second latch members extend from the body, the latch members configured to engage one another when the body is in the compacted configuration so as to restrain a portion of body from expanding to the expanded configuration. The first latch member has an axis and has a generally hook-shaped tip adapted to engage the second latch member. The first latch member is twisted about its axis while engaged with the second latch member.

In one such embodiment, the second latch member has an axis and a generally hook-shaped tip adapted to engage the hook-shaped tip of the first latch member, and the first and second latch members are both twisted about their respective axes while engaged with one another.

In another embodiment, the plurality of elastic expansile elements comprise undulating struts arranged in a circumferential ring, and the undulating struts connect to adjacent struts at apices. In some such embodiments, the tubular body is made up of a plurality of circumferential rings spaced along the longitudinal axis, and each ring is connected to at least one other ring by a plurality of longitudinal struts. In further embodiments the first and second latch members are attached to one of the plurality of longitudinal struts.

In a further embodiment, the second latch member has an axis and a generally hook-shaped tip adapted to engage the hook-shaped tip of the first latch member, and the first and second latch members are both twisted about their respective axes while engaged with one another. In some such embodiments, the first and second latch members each extend from longitudinal struts in a direction generally circumferential about the stent.

In still further embodiments, each circumferential ring has a plurality of first latch members and a plurality of second latch members, and each of the first and second latch members extend from longitudinal struts in a direction generally circumferentially about the stent so that a first latch member on a first longitudinal strut is directed toward and adapted to engage a second latch member extending from a second longitudinal strut, and the second longitudinal strut is adjacent the first longitudinal strut.

In yet another embodiment, each longitudinal strut connects to the undulating struts of a ring at a first connected apex and to the undulating struts of an adjacent ring at a second connected apex, a first end of the longitudinal strut connecting to the first connected apex, and a second end of the longitudinal strut connecting to the second connected apex.

In some embodiments, when first and second latch members are engaged, the associated first and second longitudinal struts are drawn toward one another, and undulating struts between the first and second longitudinal struts are drawn toward one another. In some such embodiments, a space is disposed between adjacent rings in the tubular body, and the first and second latch members extend into the space.

In further embodiments, first and second latch members extend from each longitudinal strut at or adjacent the first end of the strut, and the first and second latch members are adapted to engage second and first latch members, respectively, extending from adjacent longitudinal struts. Some embodiments additionally comprise a longitudinal latch member extending from the second connected apex, the longitudinal latch member adapted to overlap engaged first and second latch members. In some such embodiments, the longitudinal latch member comprises an elongate rod of sufficient length to be tuckable under engaged first and second latch members.

In still another embodiment, each hook-shaped latch member comprises a shaft portion, a crook portion and a tip portion, and the latch member is curved in the crook portion. In yet another embodiment, the latch members have a thickness, and the thickness is greater than a distance between the tip portion and the shaft portion when the latch member is in a relaxed state.

In another embodiment, a stent is combined with a balloon catheter having an inflatable balloon, and the first and second latch members are engaged so as to hold the stent in a compacted configuration on the deflated balloon. In some such embodiments, the balloon and stent are configured so that, when the balloon is inflated to a threshold diameter, balloon inflation forces deform the latching members sufficient so that all of the latching members are disengaged.

### BRIEF DESCRIPTION OF THE DRAWINGS

FIG. 1 is a perspective view of a portion of stent configured in accordance with an embodiment of the present invention.

FIG. 2 is a side view of the stent of FIG. 1.

FIG. 3 is an end view of the stent of FIGS. 1 and 2.

FIG. 4 shows a larger portion of the stent of FIGS. 1-3 depicted in a two-dimensional structural pattern view.

FIG. 5 shows a perspective view of a portion of the stent of FIGS. 1-4 with one pair of latch members engaged.

FIG. 6 is a plan view of a portion of the stent of FIGS. 1-4, showing a close-up two-dimensional depiction of latch members.

FIG. 7 is a perspective view showing a balloon catheter with an embodiment of a stent disposed on a balloon of the catheter in a collapsed configuration.

FIG. 8 shows an apparatus for loading and collapsing a stent onto a catheter.

FIG. 9 shows a portion of the apparatus of FIG. 8 showing the stent being loaded onto a catheter.

FIG. 10 is a two-dimensional structural pattern view showing a portion of a stent structure configured in accordance with another embodiment.

FIG. 11 is a two-dimensional structural pattern view of still another embodiment of a stent.

FIG. 12 is a close-up pattern view of the stent of FIG. 11.

FIG. 13 is a close-up view of a portion of the stent of FIG. 13 with latch members engaged.

FIG. 14 is a schematic cross-sectional representation of a diseased blood vessel having a catheter disposed therein and schematically illustrating potential positions of a stent deployed within the vessel.

FIG. 15 is a schematic end view of a stent configured to illustrate behavior of certain self-expandable stent embodiments.

FIG. 16 is a two-dimensional structural pattern view of another embodiment of a stent.

FIG. 17 is a two-dimensional structural pattern view of yet another embodiment of a stent.

FIG. 18 is a two-dimensional structural pattern view of a further embodiment of a stent.

FIG. 19 is a two-dimensional structural pattern view of a still further embodiment of a stent.

FIG. 20 is a two-dimensional structural pattern view of still another embodiment of a stent.

FIG. 21 is a two-dimensional structural pattern view of another embodiment of a stent.

FIG. 22 is a two-dimensional structural pattern view of yet another embodiment of a stent.

FIG. 23 is a two-dimensional structural pattern view of a yet further embodiment of a stent.

FIG. 24 is a two-dimensional structural pattern view of still another embodiment of a stent.

FIG. 25 is a two-dimensional structural pattern view of yet another embodiment of a stent.

### DETAILED DESCRIPTION OF PREFERRED EMBODIMENTS

The associated drawings and specification discuss aspects and features of the present invention in the context of several different embodiments of stents that are configured for use in the vasculature of a patient. Discussing these features in connection with stents provides for clarity and consistency in presenting these inventive features and concepts. However, it is to be understood that the features and concepts discussed herein can be applied to products other than vascular stents. For example, the self-restraining, self-expanding features described herein can be applied to prostheses for use elsewhere in the body, such as within the digestive tract. In fact, the principles discussed herein can be used in any application that would benefit from a supportive prosthesis that can be collapsed to a relatively small cross-sectional area and then later deployed, taking on the general shape of the surface to which it is deployed and applying an outwardly-directed force against the surface to which it is deployed. While Applicant specifically provides examples of use of these principles in accordance with medical devices and specifically stents, Applicant contemplates that other applications may benefit from this technology.

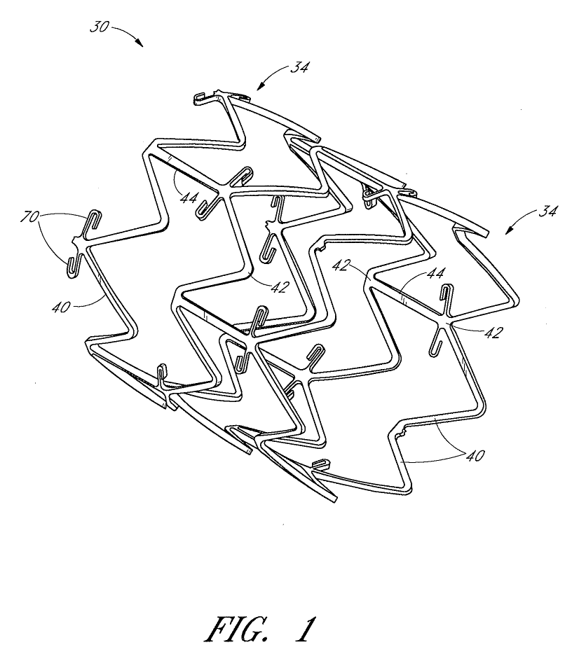

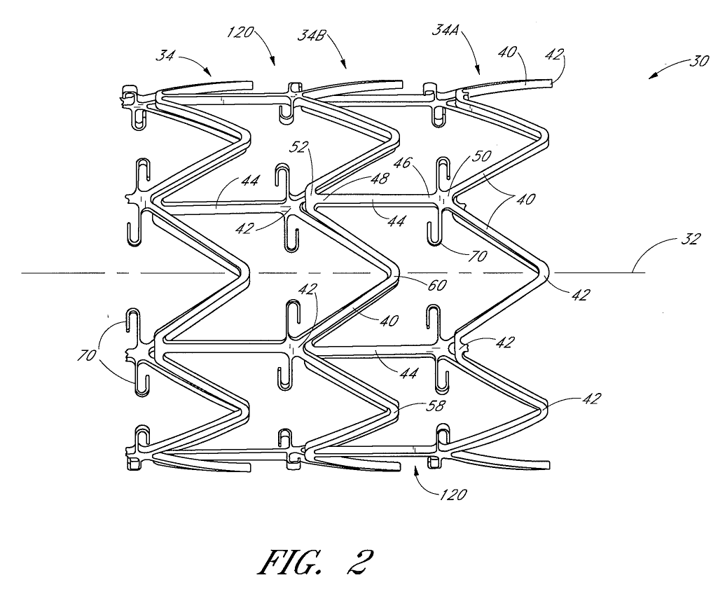

With reference initially to FIGS. 1 through 3, several views of an embodiment of a self-expanding, self-restraining stent 30 are shown. These figures depict views of the stent 30 in a relaxed, fully open position. Also, for sake of simplicity only a portion of the stent 30 is illustrated in FIGS. 1 and 2. As shown, the illustrated stent 30 preferably is tubular, with a generally circular cross-section. It is to be understood, however, that in other embodiments the tubular stent can have a non-circular cross-section, such as an oval or otherwise ovoid cross-sectional shape.

As best illustrated in FIG. 2, preferably the stent 30 is elongate and has a longitudinal axis 32. The stent 30 comprises a plurality of rows or rings 34 of circumferentially expansile elements or struts 30. The expansile elements 40 are configured to expand and contract so as to change the radial size of the stent 30. FIG. 2 shows three such rings 34; however, it is to be understood that more or fewer rings can be employed as desired to accomplish the purposes of the stent.

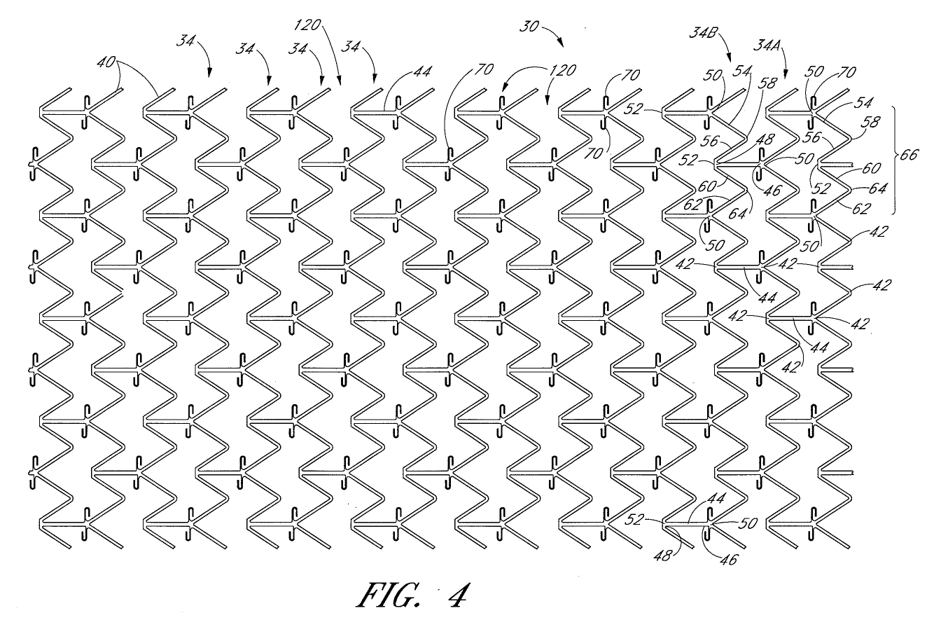

FIG. 4 depicts a much longer portion of the stent 30 of FIGS. 1-3 in a manner as if the stent 30 had been longitudinally slit and unrolled to a flattened state. Thus, FIG. 4 shows the stent 30 as a two-dimensional structural pattern, which aids in describing the stent clearly in this specification and drawings.

With continued reference to FIGS. 1-4, the respective ends of each circumferential undulating strut 40 join the adjacent strut 40 at an apex 42 which is, in at least some embodiments, an area of preferential bending. Thus each ring 34 is made up of undulating struts 40 that are connected at apices 42.

In the illustrated embodiment, each ring 34 is connected to adjacent rings 34 by one or more connecting members, or longitudinal struts 44. The longitudinal struts 44 preferably extend between apices 42 in a first ring 34A and apices 42 in an adjacent second ring 34B, as illustrated in FIGS. 1, 2 and 4. In the illustrated embodiment, the longitudinal struts 44 comprise a non-expandable rod or bar having first and second ends. The first end 46 of each longitudinal strut 44 preferably is attached to a first connected apex 50 in the first ring 34A. The second end 48 of each longitudinal strut 44 preferably is attached to a second connected apex 52 in the adjacent second ring 34B.

With continued reference to FIGS. 1 to 4, in the illustrated embodiment a first undulating strut 54 extends from a first connected apex 50 and connects to a second undulating strut 56 at a first free apex 58. The second strut 56 leads to a second connected apex 52, at which the second strut 56 connects to a third undulating strut 60 which, in turn, connects to a fourth undulating strut 62 at a second free apex 64. The fourth undulating strut 62 connects to another first connected apex 50. The first through fourth undulating struts and associated apices are referred to as a repeating expansile group 66. Preferably this structural pattern is repeated about the circumference of each ring 34. In the illustrated embodiment, each ring 34 includes four such expansile groups 66.

The strut and apex pattern in the rings of the illustrated embodiment results in a structure in which longitudinal struts 44 in adjacent rings 34 are offset relative to one another. More specifically, longitudinal struts of adjacent rings preferably are not aligned in a collinear fashion.

As discussed above, the illustrated longitudinal struts 44 are not substantially expandable in a longitudinal direction. As such, although the adjacent rings 34 are attached to one another so that the rings, as a whole, do not move substantially longitudinally relative to one another, the rings 34 still can expand and contract relatively independent of one another, and the free apices 58, 64, which are not closely connected to any longitudinal strut 44, may move flexibly in substantially any direction. As such, the individual rings 34 have substantial flexibility notwithstanding their connection to adjacent rings, and can conform to an inner surface of a blood vessel that has a changing or inconsistent diameter along its length and/or which includes plaques, necrotic tissue or other build-ups on the surface of the blood vessel.

In other embodiments, a longitudinal strut having, for instance, an undulation, spring member, or the like, can be configured to enable adjacent rings to move longitudinally relative to one another. Such structure would enhance flexibility of the stent. In still other embodiments, however, longitudinal struts between adjacent rings could be collinear with one another effectively creating a longitudinal strut that extends the length of the stent. While free apices between such longitudinal struts would still have substantial flexibility, such a configuration would reduce overall flexibility of the stent.

As best shown in FIGS. 1, 2, and 4, a plurality of latch members 70 extend transversely from each longitudinal strut 44. The latch members 70 preferably are provided in matching pairs that are configured to selectively engage one another. When engaged, the latch members 70 restrain the stent 30 in a compacted or collapsed configuration. As shown, the latch members 70 are unattached while the stent 30 is in its expanded state. However, as described in more detail below, the latch members preferably are configured to selectively engage one another so as to hold the stent in the collapsed, restrained configuration when the latch members are engaged with one another.

In the illustrated embodiment, the stent is made of a shape memory alloy, specifically nitinol. It is to be understood, however, that other materials, including metals, metal alloys, and non-metals, can be employed as appropriate.

In a preferred embodiment, the stent is initially provided as a circular-cross-section nitinol tube. The tube is laser cut according to a pattern, such as the pattern of FIG. 4 and then electrochemically polished so as to remove rough edges. The nitinol may be heat-treated as desired to obtain desired elasticity attributes.

In the embodiment illustrated in FIGS. 1 to 3, the stent 30 has been formed from a circular-cross-section nitinol tube having a constant thickness, which tube has been laser cut to the shown configuration. As such, a thickness of all of the struts 40, 44 and latching members is substantially uniform throughout the stent.

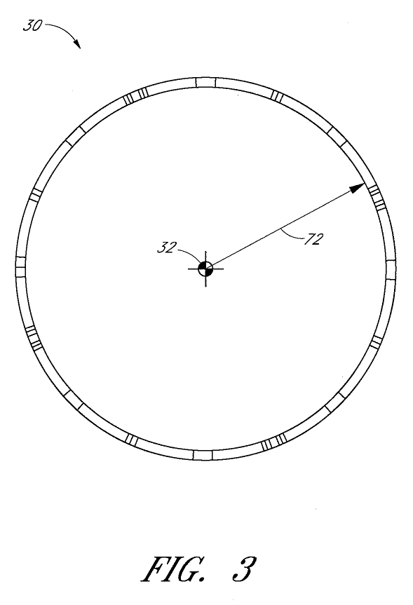

With particular reference to FIG. 3, a cross-section of the illustrated stent 30 in its fully expanded state preferably is tubular, tracking the shape of the tube from which the stent was laser cut. Thus, as cut during manufacture, all features of the stent are shaped to curve along a radius of curvature 72 around the longitudinal center axis 32. For purposes of this specification, when for instance the features of a stent fall within such a generally-uniform tubular shape, it will be referred to as a tubular plane. It is to be understood that various levels of conformance to such a tubular plane may be observed. Thus for purposes of this specification, when the members of a stent generally fall along the shape of a tube, then the stent will be considered to lie in a tubular plane. The structural pattern depicted in FIG. 4 is a two-dimensional representation of a tubular plane, specifically, the associated fully-expanded stent embodiment in its tubular plane.

With continued reference to FIGS. 1-4, in its fully-expanded shape the stent 30 is in the form of a tube having an overall thickness that is the same throughout. In configurations in which the stent 30 is not fully expanded, such as when in a collapsed state, it can be expected that at least some members of the stent will bend somewhat in directions transverse to the tubular plane. In such cases, the stent has a greater overall thickness between an innermost portion and an outermost portion than it does when fully expanded.

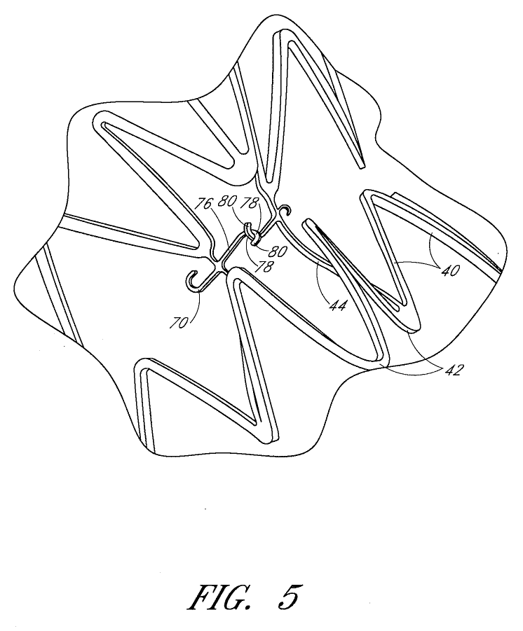

With reference next to FIG. 5, a portion of the stent 30 of FIGS. 1-4 is shown with particular focus upon one pair of latch members 70 that are engaged. As shown, engagement of the pair of latch members 70 forces the longitudinal struts 44 closer to one another, and correspondingly compresses and at least partially folds the circumferential undulating struts 40 relative to one another. As such, engagement of the latch members 70 reduces the overall circumference of the associated ring 34.

FIG. 5 illustrates a portion of a stent 30 in which only one matching pair of latch members 70 is engaged. It is thus predictable to see non-uniform deformation among components and struts of the illustrated stent. It is to be understood that when all or most of the latch members are engaged, more consistent compaction of the undulating struts about the longitudinal center axis is achieved. Particularly, when all the matching pairs of latch members in a given ring 34 are engaged, the particular ring is restrained in a compact arrangement in which undulating struts 40 are bent at their apices 42 so as to be effectively folded next to each other. Such a compacted arrangement is substantially smaller in diameter than the fully expanded, natural state of the stent.

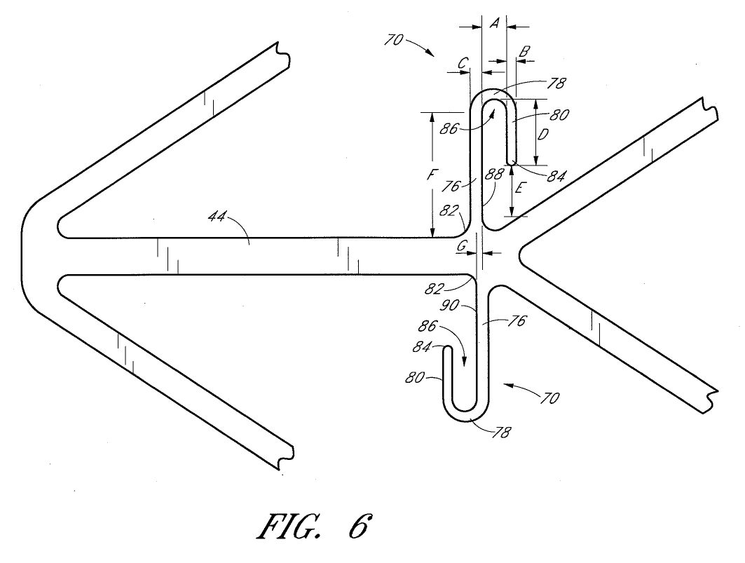

With next reference to FIG. 6, a portion of the stent 30 of FIGS. 1-5 is presented so as to show the latching members 70 in detail. As shown, the latch members 70 are generally hook-shaped, and comprise a shaft portion 76, a crook portion 78, and a tip portion 80. A base 82 of the shaft portion 76 is attached to a strut, preferably one of the longitudinal struts 44. A free end 84 of the tip 80 is spaced from other portions of the stent 30 so as to provide an entry to an engagement portion 86 defined between the tip 80, crook 78 and shaft 76.

In the illustrated embodiment, the shaft 76 is substantially straight, the crook 78 changes the direction of the latch member 70 about 180°, and the tip 80 is generally parallel to the shaft 76. It is to be understood that, in other embodiments, other configurations of latch members may be employed. For example, the shaft may by angled or curved, the crook may be configured to change the direction of the latch member less than or, in some embodiments, more than 180°, and the tip need not be parallel to the shaft. Other latch member embodiments may have a shape without a defined shaft, crook and/or tip, and still other latch member embodiments may or may not have a hook-type shape.

With additional reference again to FIG. 5, for the circumferential latch members 70 to properly engage, preferably each latch member 70 twists about 45°, and then the latch members 70 engage one another in an out-of-tubular-plane, overlapping manner in which the respective crooks 78 are disposed generally 90° relative to one another. The twisting of the latch members 70 during engagement provides a contact force enhancing the security of latch member 70 engagement. In FIG. 5, engaged latch members 70 are shown with the tip portions 80 facing outwardly. In other embodiments, the latch members 70 are engaged so that the tip portions face inwardly, thus further smoothing the outer surface of the compacted stent.

Continuing with reference to FIG. 6, preferably the tubular stent 30 has a uniform thickness as discussed above. Thus, in the illustrated embodiment, each of the longitudinal and expansile struts 44, 40, as well as the shaft 76, crook 78 and tip 80, have the same thickness. However, portions of the stent may have different widths so as to control preferential bending. As will be discussed further below, the widths of portions of the latch members 70 preferably is chosen to control both the strength and security of latch member engagement and the ease and reliability of release of such engagement so that the stent 30 can be deployed consistently and predictably.

In the illustrated embodiment, an engagement space A is defined as the distance between inner surfaces of the shaft 76 and tip 80. The tip has a width B and the shaft 76 has a width C. The length of the tip 80 is indicated by D, and an opening E is defined between the free end 84 of the tip 80 and the closest portion of the stent 30 when relaxed. The shaft 76 also has a length F defined from its connection to the strut 44 to the beginning of the crook 78. These dimensions can be manipulated in various embodiments to adjust engagement properties.

Preferably the engagement space A is greater than the shaft width C, but not as great as the thickness of the stent 30. As such, the shaft 76 will not interfere with proper engagement of the latching members 70 in a generally 90° orientation relative to one another. However, when engaged and twisted as discussed above, since the stent thickness exceeds the engagement space A, the crooks 78 of the engaged latch members 70 will be deformed so as to be partially open. This configuration provides forces to urge the latch members 70 together to remain engaged, but also facilitates further and predictable bending of the latch member 70 at and adjacent the crook 78 when it is time for deployment of the stent 30. In other embodiments the engagement space A is greater than the stent thickness.

Preferably the tip width B is less than the shaft width C so that the tip is more flexible than the shaft. As such, the tip 80 preferentially bends relative to the shaft 76 upon application of force during deployment. In some embodiments, the crook 78 has the same with as the tip 80. In other embodiments the crook 78 is wider than the tip 80, but not as wide as the shaft 76. The tip length D preferably is greater than the stent thickness and the shaft width C so as to provide a secure latch. In further preferred embodiments, the tip length D is more than twice the stent thickness so as to increase latch security during bending and moving of the compacted stent 30 when being handled by a clinician and advanced through tortuous blood vessels. The opening E preferably is greater than the stent thickness so as to allow clearance to allow the latching members to engage one another. The shaft length F can be adjusted to adjust features such as the compacted size of the stent, the ease of compaction, and the like.

In one example embodiment, a stent has a thickness of about 0.003″ and an outer, fully-expanded diameter of about 3.5 mm; the engagement space A is about 0.0025″; the tip width B is about 0.001″; the shaft width C is about 0.0015″; the tip length D is about 0.007″; and the opening is about 0.005″. Such a stent has been shown to fit upon a 2.5 mm (nominal inflated balloon diameter) balloon catheter. Of course, it is to be understood that other embodiments may have differing dimensions. Also, other stents may be configured to operate with larger balloons, including balloons sized to engage a vessel wall (such as about 3.0 mm) and balloons sized to slightly exceed the reference vessel's diameter (such as about 3.5 mm).

Continuing with reference to FIG. 6, in the illustrated embodiment the opposing shafts 76 are generally aligned with one another, but an inner surface 88 of one of the shafts 76 is offset a short distance G from an inner surface 90 of the adjacent shaft 76. Due to the offset distance G, when latch member pairs engage, their shafts will deflect. This deflection imparts a mild contact force between engaged latch member pairs. Preferably any longitudinal deflection of the latch members 70, if present, is subtle.

As the shafts 76 are generally aligned with one another, when latch member 70 pairs are engaged, forces compacting the stent 30 will be communicated circumferentially around the stent via the latch member shafts 76. Since the shafts are generally aligned as connected to the longitudinal struts 44, there will be no or minimal bending moment induced about each longitudinal strut when all latch member pairs within a ring 34 are engaged.

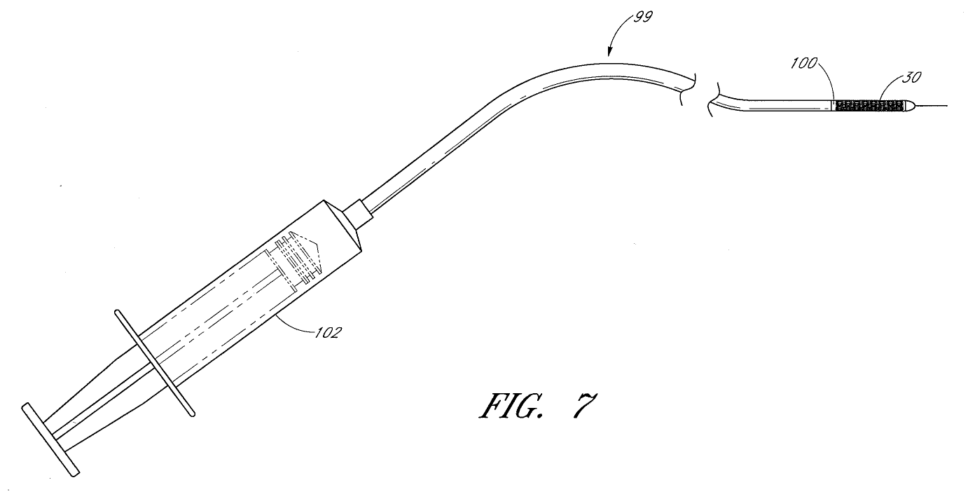

With next reference to FIG. 7, an embodiment of a treatment system is illustrated in which a catheter 99 having an uninflated balloon 100 at a distal end thereof has a stent 30 disposed in a restrained, collapsed configuration on the balloon 100. In the illustrated embodiment, the catheter 99 is sized and adapted to be negotiated through a patient's vasculature to a deployment zone. The catheter 99 is attached to a syringe 102 or other source of inflation fluid which, when actuated, pressurizes the catheter to inflate the balloon 100 and thus deploy the stent 30.

FIG. 7 illustrates an example application of the concepts discussed herein in that a stent 30 as in FIGS. 1-6 has been collapsed over the balloon 100 by engaging latch member 70 pairs, and thus reducing the outer diameter of the stent 30 to a compacted arrangement in which the stent 30 fits snugly onto the uninflated balloon 100 and is of a small enough outer diameter in its collapsed stated to be advanced through the vasculature of the patient.

Once in place on the deflated balloon 100 in a compacted, restrained configuration, the stent 30 is ready for delivery through the vasculature of a patient. During such delivery, and during handling of the device after being compacted into its restrained configuration, it can be expected that the stent 30 will be bent and squeezed somewhat. For example, in some embodiments the balloon catheter 99 will be packaged in a short capture sheath, and will endure significant handling during packaging, shipping, sterilization, and storage. Also, thermo-mechanical coupling of nitinol can cause changes in the stress of the stent retained on the balloon catheter. Further, actual use of the device in some embodiments will involve significant handling and loads such as vacuum application, removal of the capture sheath, introduction through a hemostatic valve, and traversing through a guiding catheter and tortuous artery to the treatment site. During such handling, however, the engaged latch members 70 of a preferred embodiment are securely hooked to one another. Such latch member interconnection preferably is not a rigid connection. Rather, the hook-like latch members 70 are sized and configured so that some relative movement between the latches is accommodated without causing accidental disengagement. Thus, as the balloon catheter 99 and associated compacted stent 30 is handled prior to use and while being guided into place in a patient's vasculature, the latch interconnections remain secure, holding the stent in its restrained configuration.

Once the balloon 100 has been placed at the treatment site, the stent 30 can be deployed. Preferably, deployment entails inflating the balloon 100 to a threshold size at which all of the circumferential latch member 70 pairs are disengaged, and thus the compacted undulating struts 40 are free to expand.

When the stent 30 is compacted, the engaged latch member pairs bear substantial forces to counter the outwardly-directed spring energy of the compacted undulating members 40. Such forces can be expected to deform the latch members 70 somewhat from their relaxed positions. For example, comparing the relaxed latch member 70 shape of FIG. 6 to the engaged latch members 70 of FIG. 5, it is illustrated that such forces cause the engaged tips 80 and crooks 78 to bend and deform somewhat. In one embodiment, such deformation approximates partial unraveling of the crook 78 and tip 80. When the balloon 100 is inflated, forces on the engaged latch members 70 are increased, causing further deformation of the crooks 78 and/or tips 80. Eventually the crooks and/or tips deform sufficiently that the latch member 70 disengage. The threshold size is defined as the diameter to which the balloon 100 is inflated to ensure that all of the latch members 70 disengage.

In embodiments employing two engaged hook-type latch members 70, only one or the other of the hooked latch members 70 need be deformed sufficient to release the engaged latching member pair. Thus, in the case of some manufacturing abnormality resulting, for instance, in one unusually strong latch member, such strong latch member would not prevent full release of the stent because the paired latch member likely has normal compliance, and will deform sufficient to release the stent even if the stronger latch member does not deform substantially.

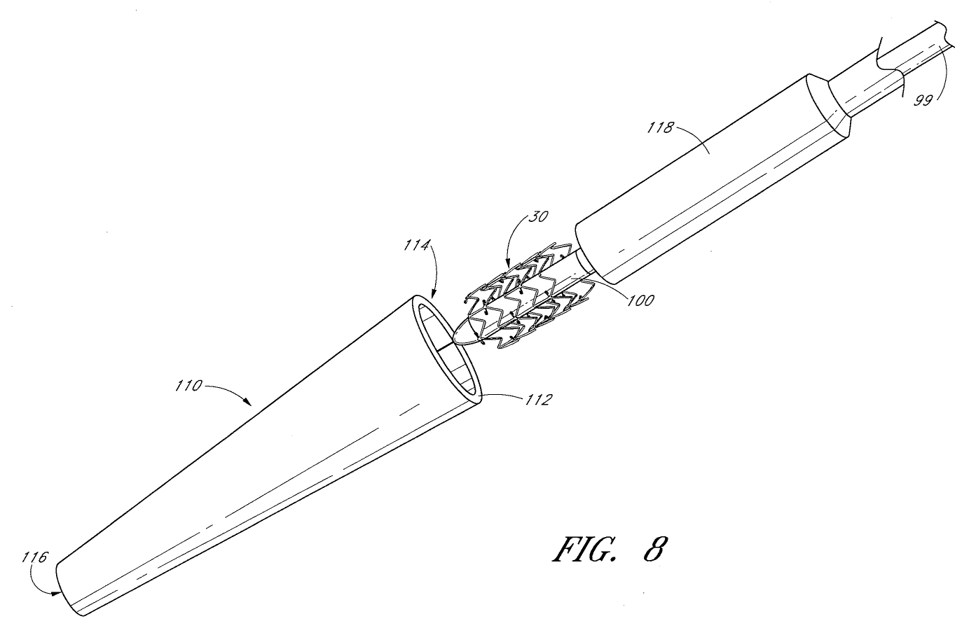

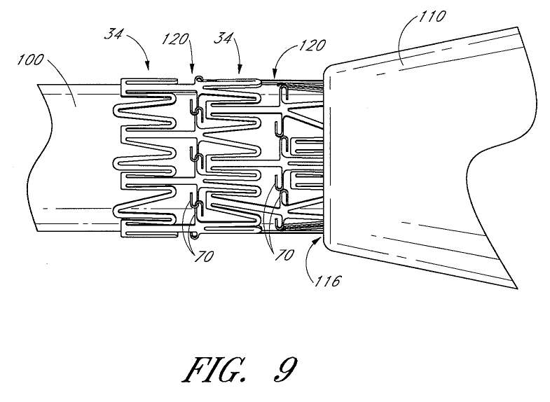

With reference next to FIGS. 8 and 9, an embodiment of a method and apparatus is depicted for collapsing a stent 30 and mounting the stent onto a balloon catheter 100. In this embodiment, a loading device 110 is generally cone- or funnel-shaped, having tapered walls 112 extending from a first, larger-diameter opening 114 to a second, smaller-diameter opening 116. The first opening 114 has a diameter larger than that of the stent's expanded diameter. In practice, an elongate member 118, such as a rubber probe, is used to push or otherwise advance the stent 30 through the funnel loading device 110. Preferably a balloon catheter 99 is advanced with the stent 30 and positioned so that when the stent 30 is compacted it will simultaneously be mounted over the deflated balloon 100. As the stent 30 moves longitudinally through the loading device 110, the tapered walls 112 compress the stent so as to fold the undulating struts 40. As such, the stent diameter is much reduced as the stent passes through the smaller second hole 116.

With particular reference to FIG. 9, as each ring 34 emerges from the second hole 116 of the loading device 110 the emerging ring 34 has been compacted by the tapered walls 112 of the loading device 110 so that the latching members 70 are very close to one another. Preferably, an operator then manually engages the latch member 70 pairs, preferably using tweezers or the like, and preferably being guided by a microscope or other optical viewing device. The stent 30 preferably is advanced incrementally through the loading device so that the latch members 70 are engaged with one another one ring at a time.

In the illustrated embodiment, the emerging rings 34, when latched together, hold snugly onto a deflated balloon 100 of the balloon catheter 99. Thus, tension between the collapsed stent 30 and the balloon 100 helps keep the stent in place. In other embodiments, the stent is collapsed independently of the balloon, and a completely-compacted stent is later slidably advanced over a deflated, catheter-borne balloon.

With continued reference to FIG. 9, the illustrated latch members 70 are positioned and adapted so that, when latch member pairs are engaged and the associated ring 34 is compacted, the undulating struts 40 do not contact and/or overlap the engaged latch members 70, or other stent structures. More specifically, in the illustrated embodiment the rings 34 are spaced apart sufficiently so that a channel 120 is provided between adjacent rings 34, and the latch member 70 pairs are provided in the channel 120. As such, the latch members 70 engage one another without interfering with the undulating struts 40 of adjacent rings 34. Notably, the rings are spaced apart sufficient to maintain the channel 120 even when the struts 40 are in a folded, compacted configuration in which the struts are more closely aligned with the stent longitudinal axis 32, and thus a greater portion of each strut's length is directed longitudinally.

Nitinol tends to form an oxide layer on its surface, which oxide layer tends to protect the nitinol from corrosion during use, particularly when deployed in the human body. The channel space 120 discussed above helps prevent load-bearing struts from overlapping, engaging, and rubbing against one another during compaction of the stent. Thus, it is less likely that the surface oxide layer of the nitinol struts will be scratched during compaction, handling, and/or deployment of the stent. Such scratched nitinol surface oxides often have difficulty growing back in vivo. Preventing scratching and wearing of load-bearing portions of the stents protects the struts from damage and ensures durability and continuing biocompatibility of the stent.

In another embodiment the stent may be designed so that at least a portion of the apices of the expansile struts is overlapped by engaged latch member pairs, thus holding the stent in an even more secure and compact configuration. Preferably the configuration is adapted so that forces between the latch members and struts are mild enough to minimize the likelihood of extensive oxide removal upon handling or deployment.

Minimizing rubbing or the like of portions of the stent can also be advantageous in drug-eluting embodiments in which the stent is coated with medication that inhibits neointimal growth, as the drug coating on the stent is largely undisturbed during handling and deployment.

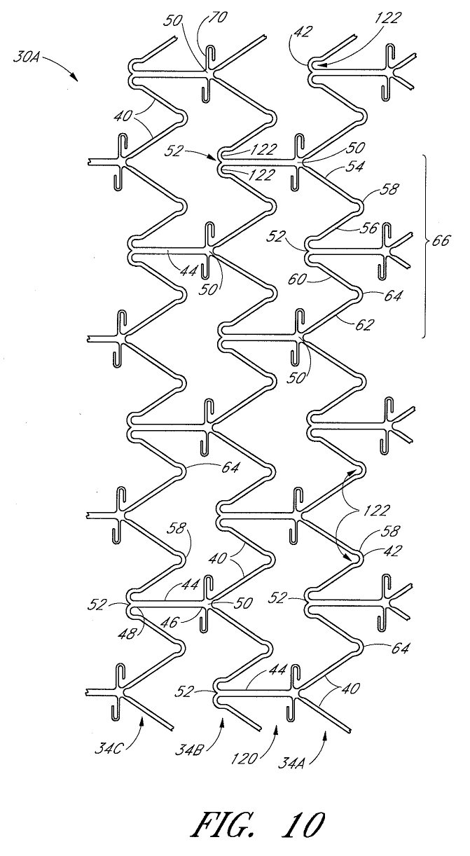

With reference next to FIG. 10, another embodiment of a tubular self-restraining, self-expanding stent 30A is presented as a 2-dimensional structural pattern representing a tubular plane. As in previous embodiments, the elongate stent comprises a plurality of circumferential rings 34 made up of undulating struts 40 joined end to end at strut apices 42. In the illustrated embodiment, the apices 42 include bending portions 122 which are configured with a radius so as to efficiently distribute bending forces through the bending portion 122 of the apex 42.

As illustrated in FIG. 10, although the entire stent preferably has substantially the same thickness, widths of particular members may vary. For example, in the illustrated embodiment, the width of the circumferential expansile struts 40 is less than the width of the longitudinal struts 44. As such, the expansile struts 40 will preferentially bend prior to the longitudinal struts 44 when the stent is subjected to stresses, specifically compaction stresses. Also, the width of the expansile struts 40 is greater than the width of the hooked latch members 70. Controlling the width of the hooked latch members enables a designer to control the force required to deform one or both of the interconnected latch members sufficient to release the connection. In the illustrated embodiment, the latch members 70 are not as wide as the expansile struts 40, and thus will preferentially bend relative to the struts.

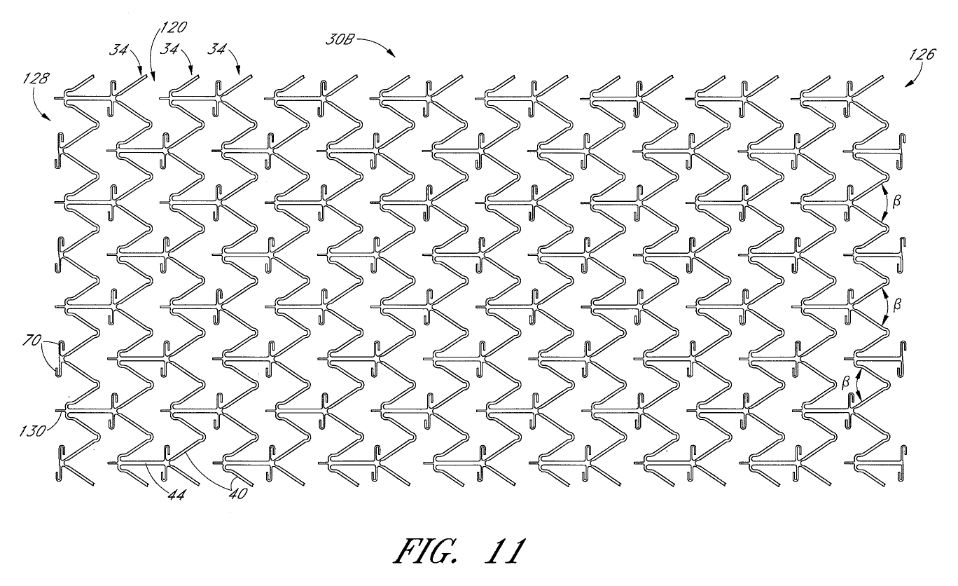

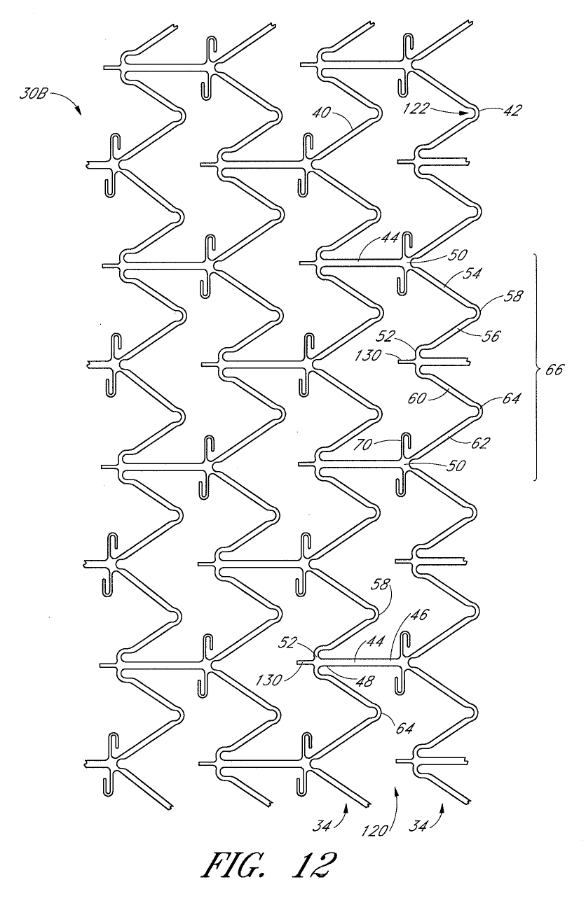

With reference next to FIGS. 11-12, yet another embodiment of a tubular self-restraining, self-expanding stent 30B is presented. FIG. 11 presents a two-dimensional structural pattern depiction of the entire stent 30B. Notably, this design depiction shows the entire length of one embodiment of such a stent. As shown, preferably both a first 126 and a second end 128 of the stent terminate at or adjacent the location of latch members 70. Thus, in this embodiment, when the stent 30B is compacted, both the first and second ends 126, 128 will be securely and directly held in place by engaged latch members 70. In other embodiments, one or both ends 126, 128 of the stent 30B may be disposed at locations spaced somewhat from latch members 70 so that free apices 58, 64 of undulating struts 40 are disposed at the ends.

With particular reference to FIG. 12, which is a close-up structural pattern view of a portion of the stent 30B of FIG. 11, some apices 42 of the rings 34 comprise a longitudinal latch member 130. In the illustrated embodiment, the longitudinal latch member 130 comprises a rod or bar that extends from an apex 42 into the channel 120 between rings 34. In the illustrated embodiment, the longitudinal latch member 130 is coaxial with a corresponding one of the longitudinal struts 44, and such longitudinal latch members 130 are not provided on free apices 58, 64 that are not directly connected to a longitudinal strut 44. It is to be understood, however, that other embodiments may employ longitudinal latch members of varying configurations and placed in various locations.

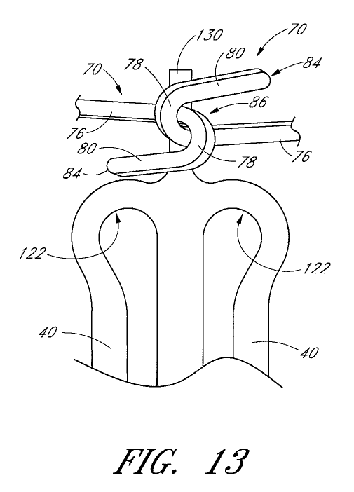

In this embodiment, when the stent 30B is placed in its compacted, restrained condition in which the circumferential latch members 70 engage one another, the longitudinal latch members 130 are tucked under engaged pairs of circumferential latch members 70 as depicted in FIG. 13. This aids in improving the compactness of the collapsed stent 30B along its length.

During extensive research and development of stents having features as described herein, Applicant noted certain behavior of stents when engagement of latch members 70 forced rings 34 into a compact state. Specifically, although portions of the rings at and adjacent the engaged latch members 70 were drawn radially inwardly, other portions of the rings would tend to flare outwardly. For example, FIG. 10 shows three rings, A, B, C of undulating struts interconnected by longitudinal struts 44 each having first and second ends 46, 48. The latch members 70 are disposed at or adjacent the first ends of the longitudinal struts 44. Thus, when the latch members 70 are engaged, the first ends 46 of the longitudinal struts 44 and the adjacent first connected apices 50 are drawn radially inwardly. However, the second ends 48 of the longitudinal struts 44 are spaced from the compacting force at the first ends 46. Thus the second ends 48 tend to resist compaction, and instead flare radially outwardly relative to the first ends 44. Such out-of-tubular-plane twisting increases the outer diameter of the collapsed stent 30A, and can thus reduce its suitability for use in small-diameter, tortuous vessels.

Continuing with reference to FIG. 10, Applicant has noted that for such a stent design the apices 52 connected to the second ends 48 of the longitudinal struts 44 tended to flare outwardly more than the free apices 58, 64.

The embodiment depicted in FIGS. 11-13 addresses and resolves much of the flaring that can occur, as it modulates the out-of-tubular-plane twisting that results from application of compacting forces to only the first end 46 of the longitudinal struts 44. More specifically, by tucking a longitudinal latch member 130 underneath engaged circumferential latch members 70, the second end 48 of each longitudinal strut 44 is secured substantially at the same radial position as the first end 46 of the longitudinal strut 44. Since compression forces are generally equalized along the length of each ring 34 (via the longitudinal struts 44), there is less out-of-tubular-plane twisting and flaring—even of expansile struts 40 and free apices 58, 64 that do not include longitudinal latch members 130. By drastically reducing flaring, configurations as depicted in FIGS. 11-13 improve the compactness and reduce the outer diameter of the stent 30B when in a collapsed configuration.

Additionally, in the illustrated embodiment, the longitudinal latch member 130 extend from an apex 50, and have no circumferential load-bearing role once the stent 30B is deployed. Thus, even if the longitudinal latch members 130 are in some way damaged prior to or during deployment of the stent 30B, once the stent is deployed, and any damage to a longitudinal latch member 130 would have minimal if any adverse effect on the stent.

With continued reference to FIGS. 11-13, preferably the longitudinal latch members 130 extend into the channel 120 between rings 34 sufficient to generally overlap engaged circumferential latch members 70. As discussed above, it is anticipated that a collapsed stent will be subjected to bending and other forces during handling. During such bending and movement, the longitudinal latch member 130 likely will move or slide relative to the engaged circumferential latch members 70. In a preferred embodiment, a length of the longitudinal latch member 130 is sufficient so that the latch member will not easily become untucked from the circumferential latch members during such handling, but not so long as to cross the channel 120 and interfere with apices or undulating struts of an adjacent ring.

With reference next to FIG. 14, an example of operation of a stent 30 within a blood vessel is depicted schematically as a cross-section. As shown, reference 140 represents what would be the reference vessel's interior surface if there were no plaques and/or lesions narrowing the vessel. Notably, such a surface 140 may be substantially circular in cross-section, or as shown here, not quite circular. A plaque/lesion 142 within the illustrated vessel significantly narrows the vessel. Reference 144 is a cross-sectional representation of the compacted stent mounted on a balloon and positioned for deployment. To deploy the stent, the balloon 100 is inflated, thus defeating the engaged latching members and releasing the stent from restraint. Reference 146 represents the balloon threshold diameter necessary to ensure full release of the stent 30. Full release is when all latch member pairs are disengaged. Reference 148 represents the fully-expanded, relaxed size of the stent. Notably, 148 preferably is greater than 140. Preferably, 148 is between about 0.2-1 mm greater in diameter than 140, and more preferably is about 0.5 mm greater in diameter than 140.

Once the stent is enlarged beyond the threshold 146, all of the latch members are disengaged. Once free of restraint the stent is outwardly self-expansile. In the illustrated embodiment, when deployed the stent 30 engages the plaque 142 and/or lesion as it expands, and expansion forces of the stent urge the plaque outwardly, thus increasing the vessel flow area. However, due to resistance of the plaque and/or lesion, and in some cases the vessel itself, the stent likely will not be capable of reaching its fully-expanded size 148 when initially deployed. As such, the initially-deployed stent will likely initially reach an intermediate size and position 150 between the threshold diameter 146 and the fully-expanded diameter 148.

With continued reference to FIG. 14, due to the flexibility of the rings, including their flexibility relative to one another, the self-expanding stent will conform to the shape and size of the vessel, maintaining substantially complete apposition while applying an outwardly-directed force urging the vessel to become and/or remain open for blood flow. Also, even if conditions within the vessel change over time, such as resolution of the thrombus, positive vessel remodeling, or the like, the stent maintains its self-expansile character, and will expand to fill spaces as they open. As such, proper apposition of the stent will be maintained even as the vessel changes over time.

In some embodiments the deployment balloon is sized and adapted so as not to substantially directly impact the vessel wall. Rather, the deployment balloon expands the stent to or beyond the threshold size 146 so as to ensure all latch members are disengaged and to release the stent from its compacted configuration, thus allowing the stent to self-expand into place without the balloon interfering with or treating plaques or the like. In one preferred embodiment, such a balloon having an operating pressure less than about 12 atmospheres may be used. It is anticipated that several types and configurations of balloons will be acceptable for deploying the stent.

In other embodiments the deployment balloon is sized and adapted not just to deploy the stent, but also to perform treatment. For instance, in one embodiment, the balloon is a non-elastic balloon having a diameter selected to match or approach the diameter 140 of the reference vessel. As such, during the same inflation cycle in which it expands and deploys the stent, the balloon also engages and displaces plaques 142 and/or other matter within the vessel so as to further enhance blood flow. In other embodiments, an elastic balloon may be employed so as to enhance blood flow but impart less trauma to the vessel. In these embodiments, when the balloon is removed, the self-expanding stent remains, conforming itself to the surface of the blood vessel and exerting a force to maintain the vessel in an opened state.

In still further embodiments the balloon is sized slightly greater than the reference vessel diameter 140, and in some embodiments greater than the stent's fully-expanded diameter 148, so as to more-dramatically treat the vessel. This procedure may also assist in even more complete engagement of the stent as the vessel elastically recoils onto the expanded stent, thus slightly compressing the stent.

With reference to FIG. 15, another aspect of certain embodiments is illustrated. As discussed above, a preferred stent embodiment is formed by laser cutting a nitinol tube. Thus, in its fully-expanded configuration, the entire stent lies within a tubular plane having a fully-expanded radius 152. However, as demonstrated schematically in FIG. 14, the stent may not reach its fully-expanded configuration when actually deployed within a diseased vessel. Instead, the stent may engage tissues within the vessel which restrain the stent from further expansion so that the stent reaches an intermediate configuration having an intermediate radius 154, which is less than 152.

Notwithstanding the limited expansion of the stent as a whole, the latch members remain biased to expand to their fully-expanded curvature, which is based on radius 152. Also, the crook portions of the latch members are not attached to any circumferentially undulating strut that may tend to temper such outward bias. Thus, in some embodiments, the crook portions of the latch members, when the stent is limited to an intermediate expansion configuration, are biased outwardly a distance 156, which is greater than intermediate radius 154.

Yet further, even when the stent 30 is expanded only to an intermediate expansion, the latch members 70 do not cross a tubular plane defined along the inner surface of the stent 30. More specifically, the released latch members are biased radially outwardly toward the blood vessel wall, and thus have minimal, if any, effect on blood flow through the artery and stent. Instead, the outwardly-biased latches may enhance engagement with the vessel wall and provide support scaffolding for the vessel. Also, even at only intermediate expansion, the stent has no overlapping portions, and maintains its nominal thickness. The ability of the stent to remain very thin, without overlapping members, is a strength of this embodiment, and helps avoid thrombus formation.

With reference next to FIG. 16, yet another embodiment of a stent structural pattern is depicted as a two-dimensional representation. In this embodiment, most or all of the apices 42 include a longitudinal latch member 130, 160. Preferably, the longitudinal latch members are positioned so that they can be tucked under engaged circumferential latch members 70. In the illustrated embodiment, several of the longitudinal latch members comprise rods or bars that extend in a direction toward the location at which the associated circumferential latch members 70 will be when engaged. In particular, the longitudinal latch members 160 that are not equidistant from both associated circumferential latch members are angled relative to the stent center axis. In other embodiments, all of the longitudinal latch members are generally parallel to the stent center axis.

In the illustrated embodiment, three longitudinal latch members 130, 160 are tucked under each engaged circumferential latch member 70 pair. Of course, it is to be understood that various shapes and sizes of longitudinal latch members, and various configurations and stent patterns, may be employed. Also, in some embodiments, one or more longitudinal latch members may be provided in pairs and, for example, be fully or mildly hook-shaped. In other embodiments, longitudinal latch members may be configured to be tucked under or over an apex of an adjacent ring.

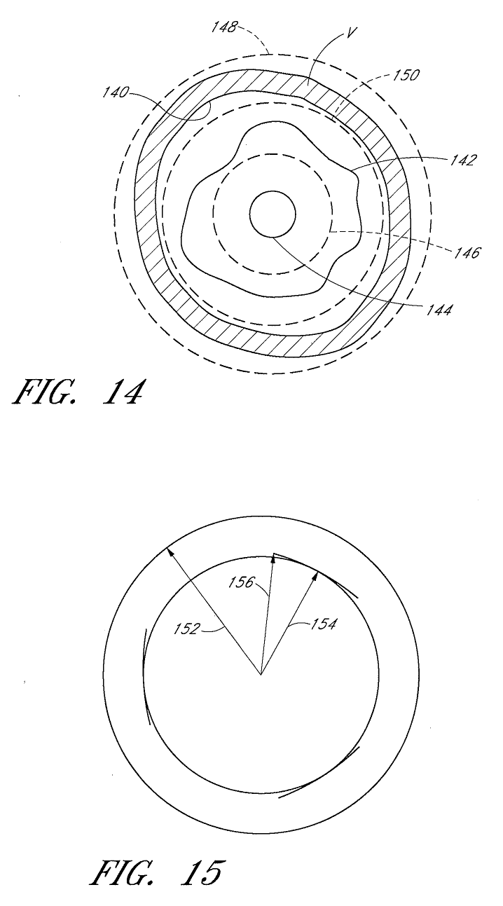

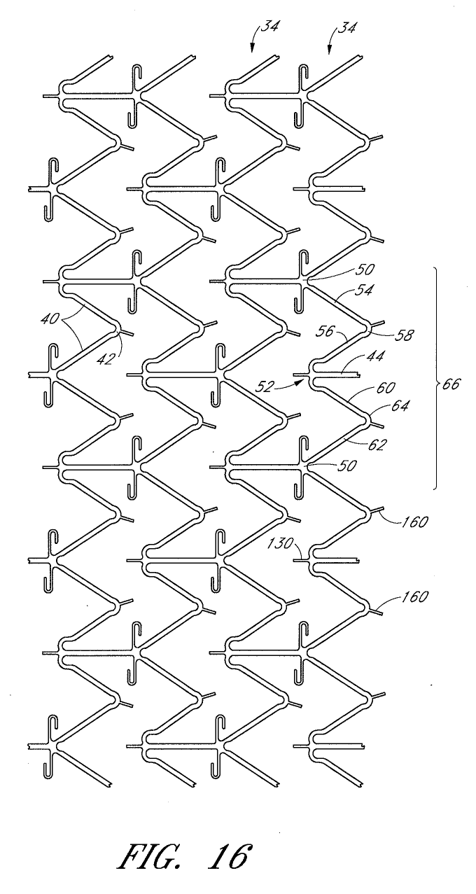

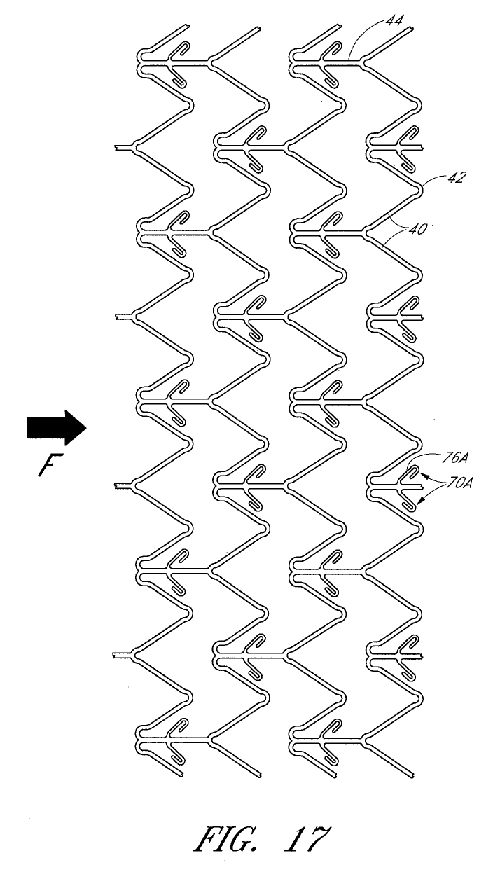

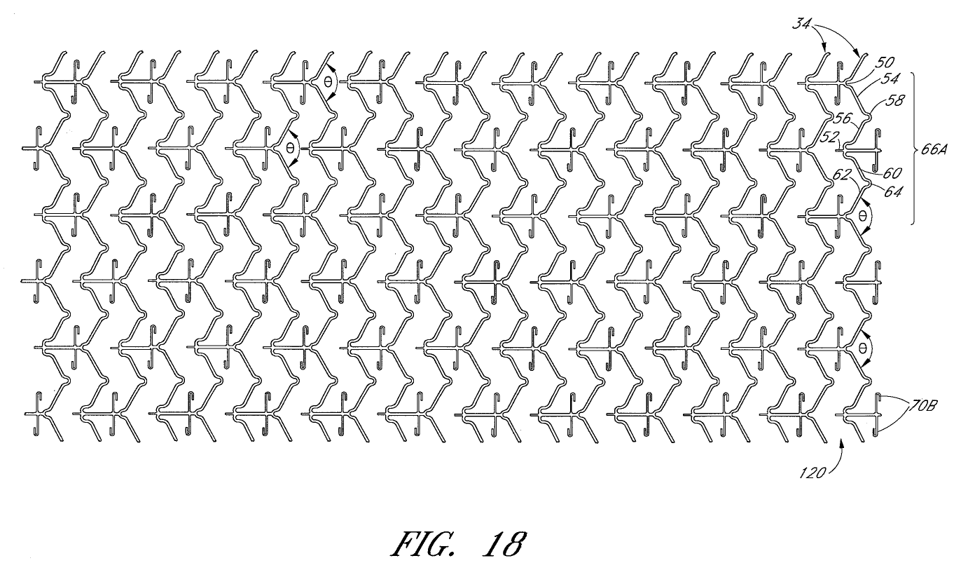

With reference next to FIG. 17, yet another stent embodiment is depicted in which the shafts 76A of the hook-like latch members 70A are generally parallel to adjacent undulating expansile struts 40. In a preferred embodiment, this stent is placed in a vessel so that blood flow F generally follows the direction of the arrow across the stent. As shown, the latch member shafts 76A are not disposed generally 90° relative to blood flow F, but preferably extend between about 30°-60° relative to the flow F, and more preferably extend about 45° relative to the flow F. It is believed that the angled latch member 70A alignment relative to blood flow F may be more beneficial hemodynamically within the vessel so as to further resist thrombogenesis.

In FIG. 17, the latch members 70A are disposed generally midway between the first and second ends 46, 48 of the longitudinal struts 44. Such placement further minimizes any bending moment between the rings 34 when the stent is compacted. More specifically, disposing the latch members 70A more centrally helps to reduce out-of-tubular-plane flaring of expansile struts 40 and associated apices 42 when the stent is compacted. Still further, such placement of the latching members 70A enables the latching members to cross over the expansile struts 40 when the stent is compacted. As such, in this embodiment the engaged latching members directly restrain the expansile struts.

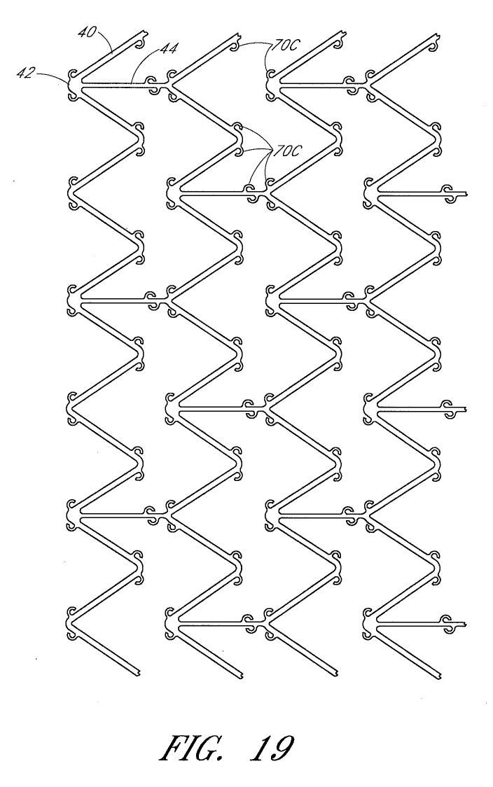

With reference next to FIG. 18, yet another stent embodiment is depicted in a two-dimensional structural pattern view. In this embodiment, the latch members 70B are disposed along the longitudinal strut 44 and spaced a relatively short distance from the first end 46 of the longitudinal strut 44. However, the latch members 70B are still placed within a channel space 120 between adjacent rings 34, and thus preferably do not interfere with or overlap the expansile struts 40 when the stent is compacted. Longitudinal latch members 130 in this embodiment help reduce flaring.

In the illustrated embodiment, the number of repeating expansile groups 66A is reduced as compared to some other embodiments, such as the embodiment shown in FIGS. 11-12, which shares some similarities with this embodiment. Specifically, the FIG. 11 stent 30 employed four expansile groups 66 (and thus four circumferential latch member 70 pairs) per ring, while the FIG. 18 stent employs only three expansile groups 66A, and three latch member 70B pairs, per ring. Also, an apex angle θ between undulations of the FIG. 18 stent is greater than an apex angle β between undulations of the FIG. 11 stent.

By employing a larger apex angle θ, the illustrated stent tends to exert greater radially-outwardly-directed forces than a similar embodiment having a lesser apex angle. Also, since the illustrated stent employs fewer latch member 70B pairs, a greater proportion of those forces are borne by each engaged latch member pair when the stent is compacted. As such, each engaged pair must bear a greater force to maintain the stent in a full or partially-collapsed state.

Preferably, embodiments are designed selecting the number of latch member pairs so that, even when most but not all of the latch member pairs in a given ring have released due to balloon inflation, expansion of the released expansile struts is not sufficient enough to accommodate balloon expansion without also releasing the remaining engaged latch members. As such, in the illustrated embodiment, all of the latch member pairs in each ring must release in order for the stent to accommodate balloon expansion to the threshold size. This configuration helps ensure complete latch member release at deployment.

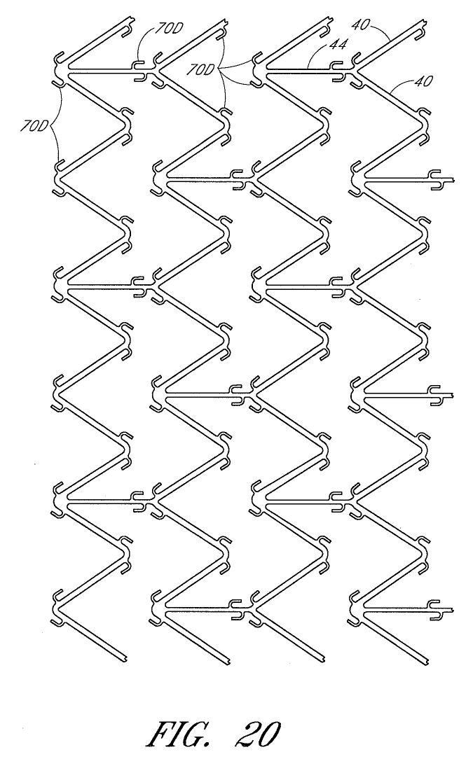

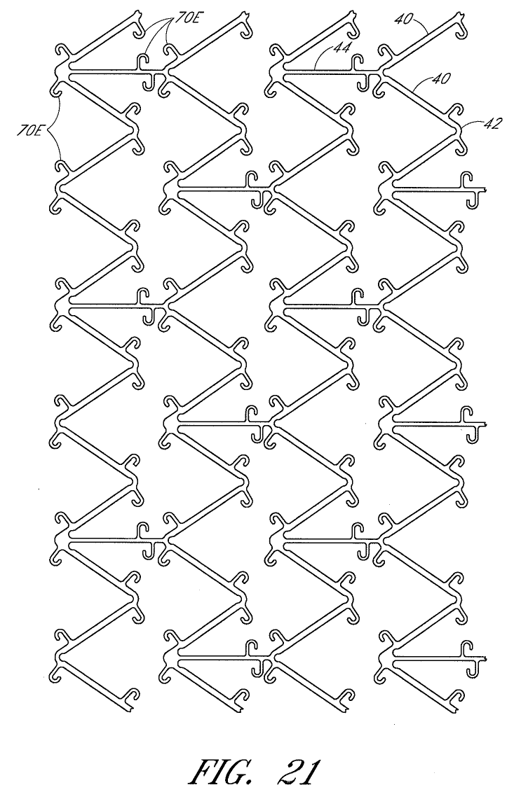

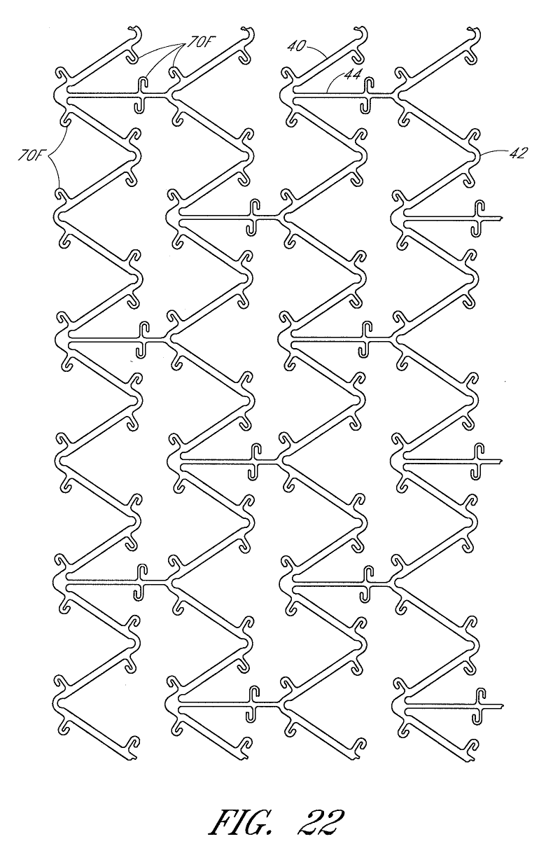

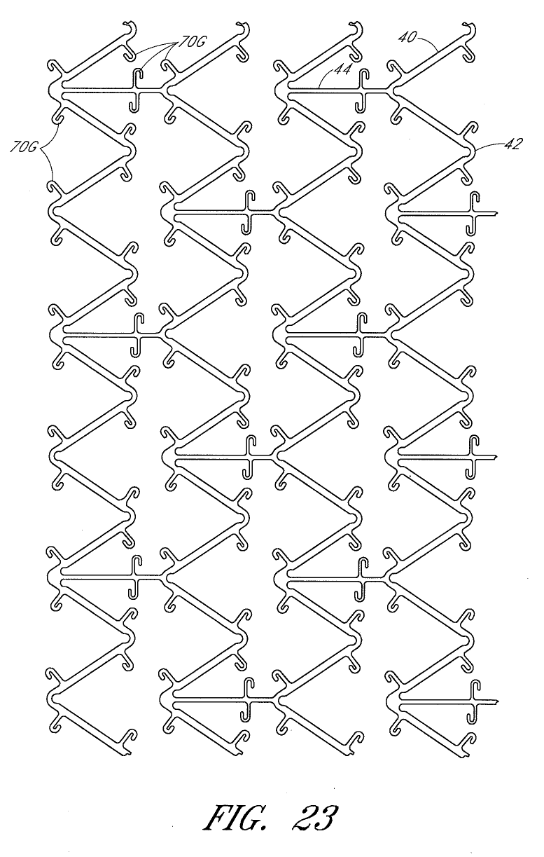

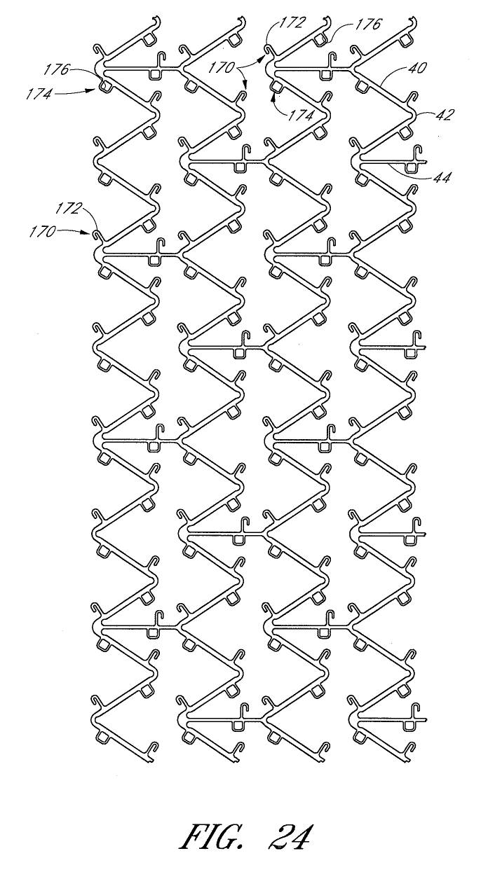

FIGS. 19-25 show schematic, two-dimensional structural pattern representations of still further embodiments employing various latch member and strut configurations. For example, FIG. 19 employs small latch members 70C disposed at or adjacent the apices 42 and at the longitudinal struts 44. FIG. 20 shows an embodiment similar to FIG. 19 but employing a more hook-like latch member 70D shape. The latches 70D of this embodiment have very little or substantially no shaft portion. Also, the latches are placed so that every undulating strut 40 can be attached to the closest adjacent strut 40, 44 upon collapsing of the stent. FIG. 21 shows an embodiment having similar placement of the latching members 70E, but having a hook-like latch member 70E construction resembling smaller versions of the general shape of the latch members 70 of, for example, the embodiment of FIGS. 1-6. FIGS. 22 and 23 show further embodiments illustrating alternative latch member 70F, 70G design and placement.

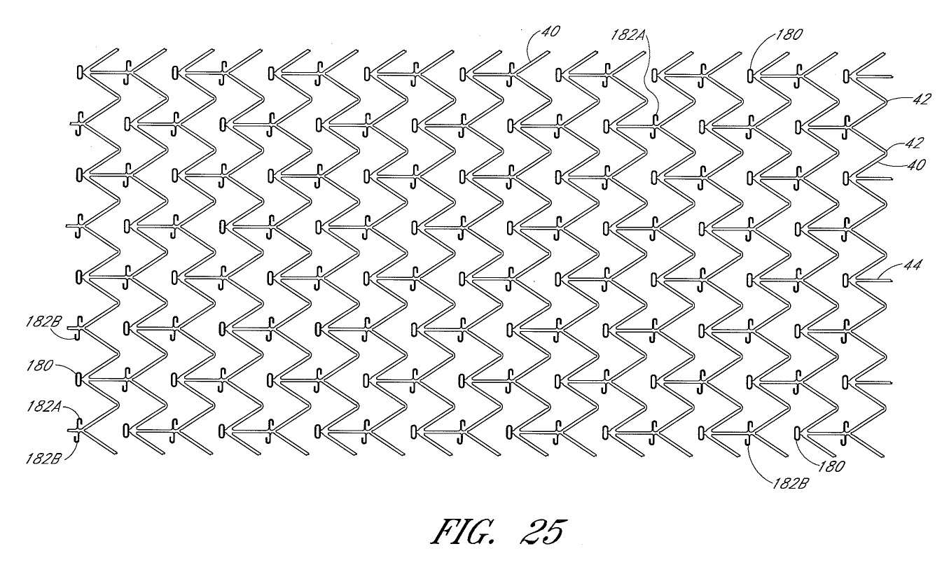

FIG. 24 illustrates another embodiment in which latch member pairs have substantially different construction. Specifically, a first one 170 of a latch member pair has a generally hook-like shape and a hooked tip 172. A second one 174 of the latch member pair defines a receiver 176 adapted to receive the hooked tip 172 of the first latch member 170. FIG. 25 illustrates another embodiment having a hook/receiver latch member pair construction, but illustrating placement of the latch members only along the longitudinal struts 44. In FIG. 25, a single receiver latch member 180 is adapted to connect to two hook-shaped latch members 182A, 182B.

The embodiments discussed herein have employed distinct circumferential rings 34 of undulating expansile struts 40 that are connected to adjacent rings by longitudinal struts 44 that are generally parallel to a longitudinal axis 32 of the stent 30. It is to be understood that, in other embodiments, other structure can be employed for connecting adjacent rings, and such structure will not necessarily have structure that prevents significant longitudinal stretching of the longitudinal strut. Further, other embodiments may employ longitudinal struts that are not necessarily parallel to the longitudinal axis, and are not necessarily straight.

Also, other embodiments may employ different configurations of the undulating expansile struts. For example, instead of circumferential rings, the undulating struts may be arranged in a helical configuration in other embodiments. For purposes of discussion consistent with terms used herein, in such a helically-configured embodiment, a single “ring” shall include a segment extending 360° about the stent's longitudinal axis.

As discussed above, although the prosthesis embodiments used in the drawings and illustrations are coronary artery stents, it is to be understood that inventive aspects discussed herein can be applied to a number of endoluminal prostheses. For example, the stent embodiments described herein are specifically intended to be used throughout the coronary arteries. However, other stents or vasculature support devices may use features of the embodiments disclosed herein for differing applications and with structure appropriate for the application. For example, stents can be used in other portions of the vasculature having different size and configuration needs or can be used in the construction of endovascular grafts. Also, devices for treating other body organs, such as the intestines, can employ aspects described herein.

Further, aspects disclosed herein can be used in connection with other products. For example, in one embodiment, a replacement heart valve has a stent attached to an annulus portion of the valve. The stent has a tubular expanded structure shaped to approximate the patient's annulus. Such an annulus may have a unique shape, and an embodiment of the stent may have undulating members specifically sized and configured to approximate that shape. Thus, such an embodiment may include some undulating members that are longer than others so that the fully-expanded stent configuration is non-circular, but approximates the patient's annulus.

For delivery, the heart valve is collapsed about a balloon catheter with the stent compacted and in a self-restrained configuration with latch member pairs engaged. The catheter is advanced to a treatment site and the balloon inflated to deploy the stent. In one embodiment, the stent's threshold expansion diameter is relatively close to the heart valve annulus diameter. During deployment, the clinician first partially inflates the balloon so as to ensure the uniquely-shaped stent is aligned correctly relative to the annulus. Once proper alignment is verified the clinician fully expands the balloon so as to exceed the stent's threshold size and deploy the heart valve/stent. In such an embodiment, the stent's threshold diameter is relatively close to the annulus size so that the clinician can have the stent/valve nearly in place before the self-expanding properties of the stent come into the play.

In the embodiment just discussed, a stent can be designed to have an irregular, non-circular fully-expanded cross-sectional shape. It is to be understood that in further embodiments the widths of certain of the struts can be varied throughout the stent in order to obtain desired expansile/contractile attributes, desired stent shapes, and to affect the strength of latching of the latch members. The thickness of portions of the stent can also be varied during manufacture by, for example, extruding a tube having portions of varying thickness, and carefully positioning the tube while laser cutting.

In the illustrated embodiment the undulating rings are generally in phase with one another, with apices generally longitudinally aligned. Other embodiments may employ other configurations. For example, the undulating rings may be out of phase with one another so that the apices are not generally longitudinally aligned. Such out of phase configurations may have a range of degree. For example, adjacent rings can be 30, 45, 60, 90 or such degrees out of phase with one another. Such arrangements may provide some advantages in flexibility of the stent for particular applications and treatment needs.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or subcombinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. For example, the latch member embodiment illustrated in FIG. 6 was depicted as part of the embodiment of FIGS. 1-4. However, such latch member embodiments can be employed in embodiments such as those depicted in FIGS. 11-13. Also, longitudinal latch members as discussed in connection with FIGS. 11-13 can also be added to embodiments such as illustrated in FIGS. 1-4. Thus, it is intended that the scope of the present invention herein disclosed should not be limited by the particular disclosed embodiments described above, but should be determined only by a fair reading of the claims that follow.

What is claimed is:

1. A self-restraining, self-expanding stent, comprising;
an elongate, unitarily-formed tubular body having a longitudinal axis and comprising a plurality of elastic expansile elements, the tubular body configured to be collapsible radially inwardly into a compacted configuration, the elastic expansile elements urging the tubular body to expand radially outwardly toward an expanded configuration; and
first and second latch members extending from the body, the latch members configured to engage one another when the body is in the compacted configuration so as to restrain a portion of body from expanding to the expanded configuration, the first latch member having an axis and having a generally hook-shaped tip adapted to engage the second latch member;
wherein the first latch member is twisted about its axis while engaged with the second latch member.

2. A stent as in claim 1, wherein the second latch member has an axis and a generally hook-shaped tip adapted to engage the hook-shaped tip of the first latch member, and wherein the first and second latch members are both twisted about their respective axes while engaged with one another.

3. A stent as in claim 1, wherein the plurality of elastic expansile elements comprise undulating struts arranged in a circumferential ring, the undulating struts connecting to adjacent struts at apices.

4. A stent as in claim 3, wherein the tubular body is made up of a plurality of circumferential rings spaced along the longitudinal axis, wherein each ring is connected to at least one other ring by a plurality of longitudinal struts.

5. A stent as in claim 4, wherein the first and second latch members are attached to one of the plurality of longitudinal struts.

6. A stent as in claim 4, wherein the second latch member has an axis and a generally hook-shaped tip adapted to engage the hook-shaped tip of the first latch member, and wherein the first and second latch members are both twisted about their respective axes while engaged with one another.

7. A stent as in claim 6, wherein the first and second latch members each extend from longitudinal struts in a direction generally circumferential about the stent.

8. A stent as in claim 6, wherein each circumferential ring has a plurality of first latch members and a plurality of second latch members, and each of the first and second latch members extend from longitudinal struts in a direction generally circumferential about the stent so that a first latch member on a first longitudinal strut is directed toward and adapted to engage a second latch member extending from a second longitudinal strut, the second longitudinal strut being adjacent the first longitudinal strut.

9. A stent as in claim 8, wherein each longitudinal strut connects to the undulating struts of a ring at a first connected apex and to the undulating struts of an adjacent ring at a second connected apex, a first end of the longitudinal strut connecting to the first connected apex, a second end of the longitudinal strut connecting to the second connected apex.

10. A stent as in claim 9, wherein when first and second latch members are engaged, the associated first and second longitudinal struts are drawn toward one another, and undulating struts between the first and second longitudinal struts are drawn toward one another.

11. A stent as in claim 10, wherein a space is disposed between adjacent rings in the tubular body, and the first and second latch members extend into the space.

12. A stent as in claim 11, wherein first and second latch members extend from each longitudinal strut at or adjacent the first end of the strut, the first and second latch members adapted to engage second and first latch members, respectively, extending from adjacent longitudinal struts.

13. A stent as in claim 12, additionally comprising a longitudinal latch member extending from the second connected apex, the longitudinal latch member adapted to overlap engaged first and second latch members.

14. A stent as in claim 13, wherein the longitudinal latch member comprises an elongate rod of sufficient length to be tuckable under engaged first and second latch members.

15. A stent as in claim 8, wherein each hook-shaped latch member comprises a shaft portion, a crook portion and a tip portion, and the latch member is curved in the crook portion.

16. A stent as in claim 15, wherein the latch members have a thickness, and the thickness is greater than a distance between the tip portion and the shaft portion when the latch member is in a relaxed state.

17. A stent as in claim 8 in combination with a balloon catheter having an inflatable balloon, wherein the first and second latch members are engaged so as to hold the stent in a compacted configuration on the deflated balloon.

18. A stent as in claim 17, wherein the balloon and stent are configured so that, when the balloon is inflated to a threshold diameter, balloon inflation forces deform the latching members sufficient so that all of the latching members are disengaged.
